(19) United States
(12) Patent Application Publication
(10) Pub. No.: US 2017/0371085 A1
(43) Pub. Date: Dec. 28, 2017

(54) LIGHT GUIDE PIPE AND LIGHT EMITTING MODULE COMPRISING THE SAME

(71) Applicant: Chung Yuan Christian University, Taoyuan City (TW)

(72) Inventors: Yih-Chih Hsu, Taoyuan (TW); Shun-Chieh Yang, New Taipei City (TW)

(21) Appl. No.: 15/195,977

(22) Filed: Jun. 28, 2016

(51) Int. Cl. F21V 8/00; A61N 5/06

(52) U.S. Cl. CPC G02B 6/0006; G02B 6/0008; A61N 5/062; A61N 2005/063

(57) ABSTRACT

A light guide pipe includes: a first light guide section formed at a first end of the light guide pipe, wherein the first light guide section is straightly extended along a longitudinal axis of the light guide pipe; a second light guide section formed at a second end of the light guide pipe, wherein an outer surface of the second light guide section is a convex curved surface, an outer diameter of the second light guide section is gradually increased along a direction from the second to the first ends, and a cavity is formed at a bottom of the second light guide section; and a third light guide section formed between the first and second light guide sections, wherein an outer surface of the third light guide section is a concave curved surface, and an outer diameter of the third light guide section is gradually decreased along the direction.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to a light guide pipe and a light emitting module, and more particularly, to a light guide pipe and a light emitting module capable of improving light transmission efficiency.

### Description of Related Arts

Photodynamic therapy is a technique using energy of light for performing treatment on a human body. A light irradiating device for photodynamic therapy can utilize a light tube, a light emitting diode or a semiconductor laser diode as a light source, for emitting light with a specific wavelength. Generally, when the light irradiating device for photodynamic therapy utilizes the light emitting diode as the light source, a light guide pipe is required to cooperate with the light emitting diode for guiding light emitted from the light emitting diode to a target irradiating area. However, when the light of the light emitting diode is transmitted from a light input end to a light output end of the light guide pipe, energy of the light is decayed in the light guide pipe, such that energy of light outputted from the light guide pipe is insufficient for Photodynamic therapy. Therefore, it is an important topic for improving light transmission efficiency of the light guide pipe in related fields.

### SUMMARY OF THE PRESENT INVENTION

The present invention provides a light guide pipe, comprising a first light guide section formed at a first end of the light guide pipe, wherein the first light guide section is straightly extended along a longitudinal axis of the light guide pipe; a second light guide section formed at a second end of the light guide pipe, wherein an outer surface of the second light guide section is a convex curved surface, and an outer diameter of the second light guide section is gradually increased along a direction from the second end to the first end, wherein a cavity is formed at a bottom of the second light guide section; and a third light guide section formed between the first light guide section and the second light guide section, wherein an outer surface of the third light guide section is a concave curved surface, and an outer diameter of the third light guide section is gradually decreased along the direction.

According to an embodiment of the light guide pipe of the present invention, the light guide pipe further comprises a fourth light guide section, formed between the second light guide section and the third light guide section, wherein an outer diameter of the fourth light guide section is greater than any of outer diameters of the first light guide section to the third light guide section.

According to an embodiment of the light guide pipe of the present invention, wherein an outer diameter of the first light guide section is smaller than or equal to the outer diameter of the third light guide section.

According to an embodiment of the light guide pipe of the present invention, a convex condensing curved surface is formed inside the cavity for condensing external light.

According to an embodiment of the light guide pipe of the present invention, the light guide pipe is formed in one piece.

The present invention further provides a light emitting module comprising a circuit substrate, a light emitting unit arranged on the circuit substrate, and a light guide pipe. The light guide pipe comprises a first light guide section, formed at a first end of the light guide pipe, wherein the first light guide section is straightly extended along a longitudinal axis of the light guide pipe; a second light guide section, formed at a second end of the light guide pipe, wherein an outer surface of the second light guide section is a convex curved surface, and an outer diameter of the second light guide section is gradually increased along a direction from the second end to the first end, wherein a cavity is formed at a bottom of the second light guide section for accommodating at least a portion of the light emitting unit; and a third light guide section, formed between the first light guide section and the second light guide section, wherein an outer surface of the third light guide section is a concave curved surface, and an outer diameter of the third light guide section is gradually decreased along the direction.

According to an embodiment of the light emitting module of the present invention, the light emitting module further comprises a first base, and a second base connected to the first base, wherein the light guide pipe is fixed on the first base and the circuit substrate is fixed on the second base.

According to an embodiment of the light emitting module of the present invention, heat dissipation fins are formed on outer surfaces of the first base and the second base, and an accommodation space is formed inside the second base for accommodating a heat dissipation fan.

According to an embodiment of the light emitting module of the present invention, the light emitting module further comprises a fixing ring, wherein the light guide pipe further comprises a fourth light guide section formed between the second light guide section and the third light guide section, wherein an outer diameter of the fourth light guide section is greater than any of outer diameters of the first light guide section to the third light guide section, wherein the fixing ring abuts against the fourth light guide section for fixing the light guide pipe to the first base.

According to an embodiment of the light emitting module of the present invention, an outer diameter of the first light guide section is smaller than or equal to the outer diameter of the third light guide section.

According to an embodiment of the light emitting module of the present invention, a convex condensing curved surface is formed inside the cavity for condensing light emitted from the light emitting unit.

According to an embodiment of the light emitting module of the present invention, a light emitting angle of the light emitting unit is 60 degree.

According to an embodiment of the light emitting module of the present invention, the light guide pipe is formed in one piece.

According to an embodiment of the light emitting module of the present invention, the light emitting module further comprises a light guide unit arranged in front of the first end of the light guide pipe for changing a light traveling angle of light outputted from the first end of the light guide pipe.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram showing a light emitting module of the present invention.

FIG. 2 is a diagram showing a light guide pipe of the present invention.

### DETAILED DESCRIPTION OF THE PREFERRED EMBODIMENT

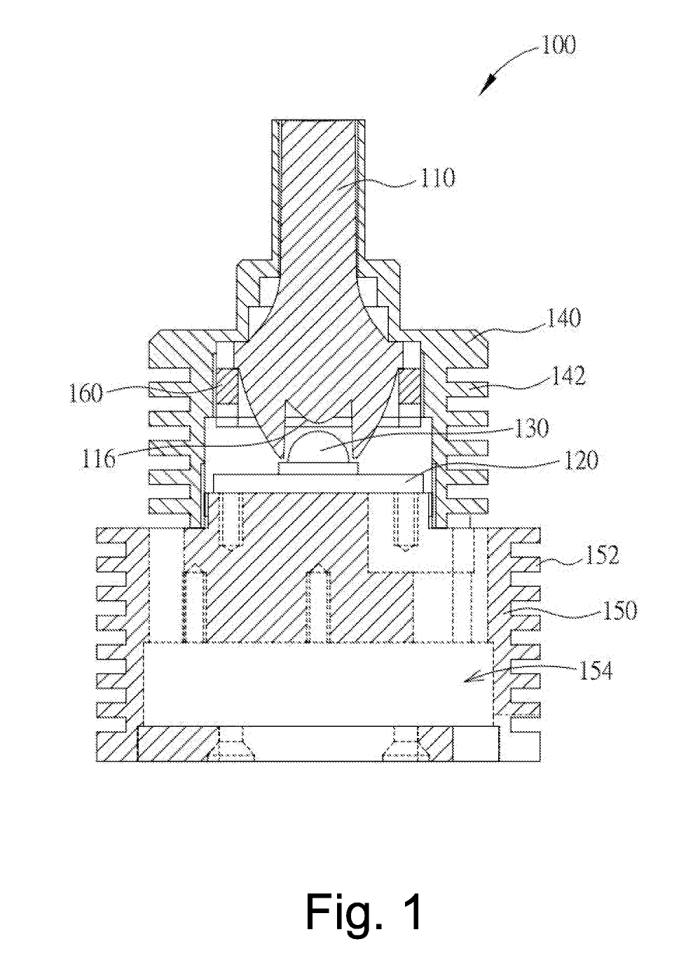

Please refer to FIG. 1. FIG. 1 is a diagram showing a light emitting module of the present invention. As shown in FIG. 1, the light emitting module 100 of the present invention comprises a light guide pipe 110, a circuit substrate 120, a light emitting unit 130, a first base 140, and a second base 150. The light emitting unit 130 is arranged on the circuit substrate 120. The circuit substrate 120 is configured to provide power to the light emitting unit 130 for driving the light emitting unit 130 to emit light. The light guide pipe 110 is configured to guide the light emitted from the light emitting unit 130. The light guide pipe 110 is fixed on the first base 140, and the circuit substrate 120 is fixed on the second base 150. The first base 140 is connected to the second base 150, and heat dissipation fins 142, 152 are formed on outer surfaces of the first base 140 and the second base 150 for dissipating heat generated by the light emitting unit 130 when emitting light. In addition, an accommodation space 154 is formed inside the second base 150 for accommodating a heat dissipation fan (not shown in the figures), in order to further discharge heat out of the light emitting module 100 when the light emitting unit 130 emits light.

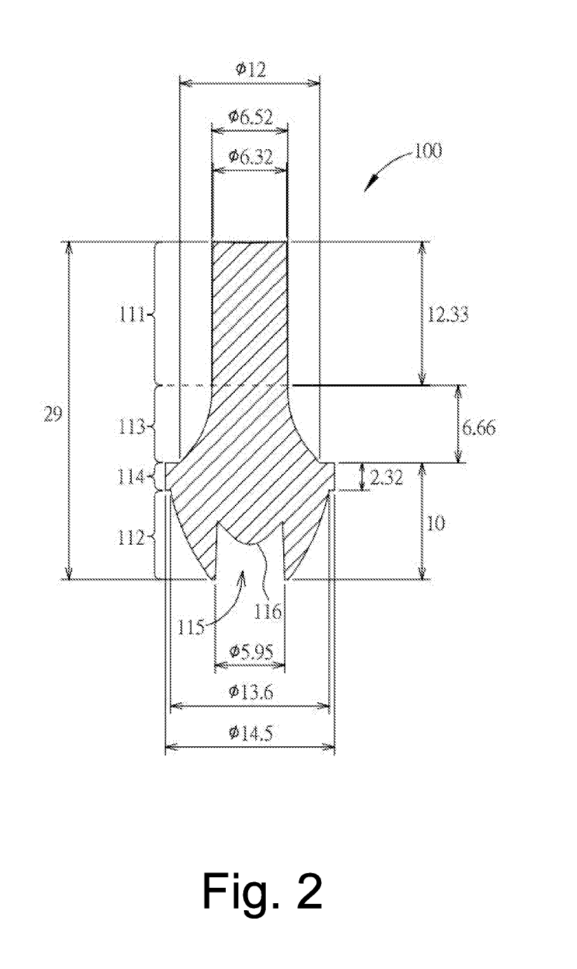

Please refer to both FIG. 2 and FIG. 1. FIG. 2 is a diagram showing a light guide pipe of the present invention. As shown in FIG. 2, the light guide pipe 110 of the present invention comprises a first light guide section 111 formed at a first end of the light guide pipe 110, a second light guide section 112 formed at a second end of the light guide pipe 110, and a third light guide section 113 formed between the first light guide section 111 and the second light guide section 112. The first light guide section 111 is straightly extended along a longitudinal axis of the light guide pipe 110. An outer surface of the second light guide section 112 is a convex curved surface, and an outer diameter of the second light guide section 112 is gradually increased along a direction from the second end to the first end of the light guide pipe 110. In addition, a cavity 115 is formed at a bottom of the second light guide section 112 for accommodating at least a portion of the light emitting unit 130. A convex condensing curved surface 116 is formed inside the cavity 115 and faces toward a light emitting surface of the light emitting unit 130. The convex condensing curved surface 116 is configured to condense light emitted from the light emitting unit 130. An outer surface of the third light guide section 113 is a concave curved surface, and an outer diameter of the third light guide section 113 is gradually decreased along the direction from the second end to the first end of the light guide pipe 110. The outer diameter of the third light guide section 113 is gradually decreased to be slightly greater than or substantially equal to an outer diameter of the first light guide section 111. In other words, the outer diameter of the first light guide section 111 is smaller than or equal to the outer diameter of the third light guide section 113.

When the light emitting unit 130 emits light, the convex condensing curved surface 116 in the cavity 115 of the second light guide section 112 receives and guides the light emitted from the light emitting unit 130 into the light guide pipe 110. The convex outer surface of the second light guide section 112 is configured to work as a reflection surface for reflecting light inside the light guide pipe 110 to the first light guide section 111, in order to prevent the light inside the light guide pipe 110 from being emitted from the outer surface of the second light guide section 112. In addition, the concave outer surface of the third light guide section 113 is configured to further collect the light inside the light guide pipe 110 to the first light guide section 111, for outputting the light from the first end of the light guide pipe 110. According to the above design, the light guide pipe 110 of the present invention has improved light transmission efficiency. For example, when the light emitting unit 130 is a 10-watt single-chip light emitting diode, power of light outputted from the first end of the light guide pipe 110 can reach 2 watts, where a light guide pipe of a conventional light emitting module can only output 0.5 watt power. Thus the light guide pipe 110 of the present invention has higher light transmission efficiency. Accordingly, when the light emitting module 100 of the present invention is applied to the field of photodynamic therapy, the light emitting module 100 of the present invention can have higher light emitting efficiency, for providing sufficient light energy.

In addition, dimensions (in millimeter) of the parts of the light guide pipe 110 are marked in FIG. 2. The light guide pipe 110 of the present invention can have better light transmission efficiency in such dimensional ratio of FIG. 2. However, the light guide pipe 110 of the present invention shall not be limited to the above dimensional ratio. Moreover, when a light emitting angle of the light emitting unit is 60 degree, a combination of the light guide pipe 110 and the light emitting unit 130 of the present invention can have better light transmission efficiency. However, the light emitting unit 130 of the present invention shall not be limited to the above light emitting angle.

On the other hand, the light guide pipe 110 of the present invention can further comprise a fourth light guide section 114 formed between the second light guide section 112 and the third light guide section 113. An outer diameter of the fourth light guide section 114 is greater than any of outer diameters of the first to third light guide sections 111-113. Moreover, the light emitting module 100 can further comprise a fixing ring 160. The fixing ring 160 is fixed to the first base 140 and abuts against the fourth light guide section 114, for clamping the light guide pipe 110 between the first base 140 and the fixing ring 160, in order to further fix the light guide pipe 110 to the first base 140. However, the way of fixing the light guide pipe 110 by the fixing ring is only an example of the present invention, and the present invention shall not be limited to the above embodiment. In other embodiment of the present invention, the light guide pipe 110 can be fixed to the first base 140 by other kind of structure as well.

Moreover, the light guide pipe 110 of the present invention can be produced by injection molding in order to reduce production cost. The light guide pipe 110 can be formed in one piece. Furthermore, the light emitting module 100 of the present invention can further comprise a light guide unit (not shown in the figures) arranged in front of the first end of the light guide pipe 110, for changing a light traveling angle of light outputted from the first end of the light guide pipe 110. For example, an original light outputting path of the first end of the light guide pipe 110 is parallel to the longitudinal axis of the light guide pipe 110, and the light guide unit can deflect the light outputting path an angle (such as 45 degrees), so as to increase the application range of the present invention. The light guide unit can be a reflection mirror or a light guide strip.

In contrast of the prior art, the outer surface of the second light guide section 112 of the light guide pipe 110 is a convex curved surface for reflecting the light inside the light guide pipe 110 to the first light guide section 111, and the outer surface of the third light guide section 113 is a concave curved surface for collecting the light inside the light guide pipe 110 to the first light guide section 111. Therefore, the light guide pipe 110 of the present invention has better light transmission efficiency. Accordingly, the light emitting module 100 of the present invention also has higher light emitting efficiency.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A light guide pipe, comprising:
a first light guide section, formed at a first end of the light guide pipe, wherein the first light guide section is straightly extended along a longitudinal axis of the light guide pipe;
a second light guide section, formed at a second end of the light guide pipe, wherein an outer surface of the second light guide section is a convex curved surface, wherein an outer diameter of the second light guide section is gradually increased along a direction from the second end to the first end, wherein a cavity is formed at a bottom of the second light guide section; and
a third light guide section, formed between the first light guide section and the second light guide section, wherein an outer surface of the third light guide section is a concave curved surface, and an outer diameter of the third light guide section is gradually decreased along the direction.

2. The light guide pipe of claim 1 further comprising a fourth light guide section, formed between the second light guide section and the third light guide section, wherein an outer diameter of the fourth light guide section is greater than any of outer diameters of the first light guide section to the third light guide section.

3. The light guide pipe of claim 1, wherein an outer diameter of the first light guide section is smaller than or equal to the outer diameter of the third light guide section.

4. The light guide pipe of claim 1, wherein a convex condensing curved surface is formed inside the cavity for condensing external light.

5. The light guide pipe of claim 1, wherein the light guide pipe is formed in one piece.

6. A light emitting module, comprising:
a circuit substrate;
a light emitting unit, arranged on the circuit substrate; and
a light guide pipe, comprising:
a first light guide section, formed at a first end of the light guide pipe, wherein the first light guide section is straightly extended along a longitudinal axis of the light guide pipe;
a second light guide section, formed at a second end of the light guide pipe, wherein an outer surface of the second light guide section is a convex curved surface, and an outer diameter of the second light guide section is gradually increased along a direction from the second end to the first end, wherein a cavity is formed at a bottom of the second light guide section for accommodating at least a portion of the light emitting unit; and
a third light guide section, formed between the first light guide section and the second light guide section, wherein an outer surface of the third light guide section is a concave curved surface, and an outer diameter of the third light guide section is gradually decreased along the direction.

7. The light emitting module of claim 6 further comprising a first base and a second base connected to the first base, wherein the light guide pipe is fixed on the first base and the circuit substrate is fixed on the second base.

8. The light emitting module of claim 7, wherein heat dissipation fins are formed on outer surfaces of the first base and the second base, and an accommodation space is formed inside the second base for accommodating a heat dissipation fan.

9. The light emitting module of claim 7 further comprising a fixing ring, wherein the light guide pipe further comprises a fourth light guide section, formed between the second light guide section and the third light guide section, wherein an outer diameter of the fourth light guide section is greater than any of outer diameters of the first light guide section to the third light guide section, wherein the fixing ring abuts against the fourth light guide section for fixing the light guide pipe to the first base.

10. The light emitting module of claim 6, wherein an outer diameter of the first light guide section is smaller than or equal to the outer diameter of the third light guide section.

11. The light emitting module of claim 6, wherein a convex condensing curved surface is formed inside the cavity for condensing light emitted from the light emitting unit.

12. The light emitting module of claim 6, wherein a light emitting angle of the light emitting unit is 60 degree.

13. The light emitting module of claim 6, wherein the light guide pipe is formed in one piece.

14. The light emitting module of claim 6, further comprising a light guide unit arranged in front of the first end of the light guide pipe for changing a light traveling angle of the light outputted from the first end of the light guide pipe.
